(19) United States
(12) Patent Application Publication
(10) Pub. No.: US 2009/0221490 A1
(43) Pub. Date: Sep. 3, 2009

(54) Endothelin Receptors In Morphine Withdrawal

(76) Inventors: Anil Gulati, Naperville, IL (US); Shaifali Bhalla, Oak Park, IL (US)

Correspondence Address: Dewey & LeBoeuf LLP, 1101 New York Avenue, NW, Washington, DC, 20005, US

(21) Appl. No.: 12/161,200

(22) PCT Filed: Sep. 8, 2006
(86) PCT No.: PCT/US2006/035169

§ 371 Date: Jul. 17, 2008

Related U.S. Application Data
(60) Provisional application No. 60/715,794, filed on Sep. 9, 2005.

(51) Int. Cl. A61K 38/17; G01N 33/53

(52) U.S. Cl. 514/12; 435/7.1

(57) ABSTRACT

The present invention relates to compositions and methods for managing opioid tolerance and reducing opioid withdrawal. More specifically, the present invention provides for endothelin, endothelin receptors, and endothelin receptor antagonists and agonists as a means for managing G-protein activity in the context of opioid tolerance and withdrawal.

### FIELD OF THE INVENTION

The present invention relates to compositions and methods for managing opioid tolerance and reducing opioid withdrawal in individuals. More specifically, the present invention provides for endothelin, endothelin receptors, and endothelin receptor antagonists and agonists as a means for managing G-protein activity in the context of opioid tolerance and withdrawal.

### BACKGROUND OF THE INVENTION

Because of reported benefits to neonatal behavior and outcomes from opioid-based analgesia and anesthesia, opioids are widely used in neonatal pain management. Chronic use of opioid analgesics results in tolerance and dependence, however. Chronic exposure of the fetus during maternal opiate abuse also leads to severe neurological and behavioral changes. Abrupt cessation of opiates leads to severe withdrawal syndrome and management of these adverse effects may be a major challenge for the clinician.

Thus, although opioids may offer beneficial therapy, there remains a need for better understanding of opioid withdrawal and compositions and methods for managing opioid withdrawal.

### SUMMARY OF THE INVENTION

An object of the present invention provides for better understanding of opioid withdrawal and compositions and methods for managing opioid tolerance and withdrawal. In particular, the present invention provides for better management of opioid tolerance and withdrawal syndrome by manipulating G-protein coupling to opioid receptors through use of ET receptor antagonists.

One embodiment if the present invention provides for a method for managing opioid tolerance or withdrawal by administering an effective amount of an ET receptor antagonist.

Another embodiment of the present invention provides a method for regulating G-protein coupling to opioid receptors in an individual experiencing opioid tolerance or withdrawal, the method comprising administering an effective amount of an ET receptor antagonist.

Another embodiment of the present invention provides a method for identifying a novel ET receptor antagonist, the method comprising assessing the G-protein coupling activity to ET receptors in a biological sample obtained from an individual experiencing opioid tolerance or withdrawal.

Yet another embodiment of the present invention provides a method for managing opioid tolerance or withdrawal, the method comprising assessing the G-protein coupling activity to ET receptors in a biological sample obtained from an individual experiencing opioid tolerance or withdrawal.

### BRIEF DESCRIPTION OF THE DRAWINGS

FIG. 1a illustrates Morphine-stimulated [³⁵S]GTPγS binding in the brain of placebo and morphine withdrawal group. Morphine produced concentration-dependent increase in [³⁵S]GTPγS binding in the brain of neonatal rats. Values are mean ±SEM; N=4.

FIG. 1b depicts ET-1-stimulated [³⁵S]GTPγS binding in the brain of placebo and morphine withdrawal group. ET-1 produced concentration-dependent increase in [³⁵S]GTPγS binding in the brain of neonatal rats. Values are mean ±SEM; N=4.

FIG. 2a illustrates ET_{A} receptor antagonist, BMS 182874-stimulated [³⁵S]GTPγS binding in the brain of placebo and morphine withdrawal group. BMS182874 produced concentration-dependent increase in [³⁵S]GTPγS binding in the brain of neonatal rats. Values are mean ±SEM; N=4.

FIG. 2b shows ET_{B} receptor agonist, IRL1620-stimulated [³⁵S]GTPγS binding in the brain of placebo and morphine withdrawal group. IRL1620 produced concentration-dependent increase in [³⁵S]GTPγS binding in the brain of neonatal rats. Values are mean ±SEM; N=4.

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Thus, for example, the reference to a antagonist is a reference to one or more such antagonists, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages may mean±1%.

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason.

Unless defined otherwise, all technical and scientific terms used herein have the same meaning as those commonly understood to one of ordinary skill in the art to which this invention pertains.

Several reported benefits to neonatal behavior and outcomes from opioid-based analgesia and anesthesia have led to the wide use of opioids in neonatal pain management. See Anand et al. 153(4) Arch Pediatr Adolesc Med 331-38 (1999); Anand et al. 1(8524) Lancet 62-66 (1987); MacGregor et al. 79(1) Arch Dis Child Fetal Neonatal Ed F40-3 (1998). Chronic use of opioid analgesics results, however, in tolerance and dependence. latrogenic opioid dependence was first reported in infants receiving fentanyl during Extra Corporeal Membrane Oxygenation (ECMO). Suresh & Anand 11(5) Paediatr Anaesth 511-21 (2001). Chronic exposure of the fetus during maternal opiate abuse also leads to severe neurological and behavioral changes. Abrupt cessation of opiates leads to severe withdrawal syndrome and management of these adverse effects may be a major challenge for the clinician. Recent animal studies have demonstrated that fetal rats and infant rat pups undergo opiate tolerance and physical dependence manifested as withdrawal, if the dams are exposed to opiates during pregnancy. See Jones & Barr 66(2) Pharmacol Biochem Behav 419-24 (2000); Jones & Barr 109(6) Behav Neurosci 1189-98 (1995); Windh et al. 273(3) J Pharmacol Exp Ther 1361-74 (1995).

Morphine and other opioids act by binding to and activating μ, κ, and δ opioid receptors. Conformational changes in opioid receptors initiate signal transduction cascade due to activation of inhibitory G-proteins, Giα and Go. See Zhang et al. 95(12) Proc Nat'l Acad Sci USA 7157-62 (1998); Suresh & Anand 22(5) Semin Perinatol 425-33 (1998); Liu & Anand 38(1-2) Brain Res Brain Res Rev 1-19 (2001). During opioid tolerance, opioid receptors are desensitized and there is an upregulation of second messengers such as AC and cAMP. Avidor-Reiss et al. 272(8) J Biol Chem 5040-47 (1997). Chronic morphine treatment also results in functional uncoupling of μ-opioid receptors and G-proteins. The invention herein provides that endothelin (ET), an endogenous neuropeptide, may be an important factor in mediating opiate tolerance.

ET is an extremely potent endothelium derived vasoconstriction factor (Hickey et al. 248 (5.1) Am J Physiol C550-6 (1985)) that was isolated, sequenced, and cloned (Yanagisawa et al. 6(4) J Hypertens Suppl S188-91 (1988)). Endothelins are 21 amino acid, highly potent vasoconstrictive peptides with two disulfide bonds. Endothelins are produced biologically by enzymatically cleaving preproendothelin to proendothelin, then to endothelin by endothelin-converting enzymes. ET exerts biological effects by binding to cell surface receptors which are 7-transmembrane receptors coupled to G-proteins. There are two distinct types of endothelin receptors, (a) the ET-1 selective ET_{A} receptors primarily found on vascular smooth muscle and responsible for vasoconstriction, and (b) nonselective ET_{B} receptors primarily found in vascular endothelium and responsible for vasodilation.

The vasoconstrictive effects of ET-1 are mediated predominantly by G-protein coupled ET_{A} receptors. Reynolds et al. 160(2) Biochem Biophys Res Commun 868-73 (1989). ET-1 also is made in high concentrations by prostate, metastatic cancers, and CNS. ET in the CNS is produced by endothelial cells and nonendothelial cells, such as neurons, astrocytes, and glial cells. MacCumber et al. 87(6) Proc Nat'l Acad Sci USA 2359-63 (1990).

The global distribution of ET and its binding sites in the brain suggests that, in addition to being a vasoconstrictor, it may be acting as an important neuropeptide in the CNS. Gulati et al. 26 Drug Develop Res 361-87 (1992). Endothelin (ET) receptor antagonists, in particular selective ET_{A} or balanced antagonists ET_{A}/ET_{B}, represent a therapeutic area for diseases such as congestive heart failure (CHF) and pulmonary hypertension. BQ-123 and BMS-182874 are specific antagonists of ET_{A} receptors. Ihara et al. 50(4) Life Sci. 247-55 (1992); Stein et al. 37(3) J Med Chem 329-31 (1994). Endothelin antagonists have profound effects on the pulmonary vasculature and the right heart, whereas ACE inhibitors primarily affect the peripheral vessel and the left heart.

Several studies indicate that the central ET receptors are predominantly of ET_{B} subtype. Matsumura et al. 17(6.2) Hypertension 1192-6 (1991). Rat cerebral astrocytes have been shown to express mainly ET_{B} type of receptors (Hama et al. 186(1) Biochem Biophys Res Commun 355-62 (1992) and glial cells also were found to intensely express ET_{B} receptor mRNA (Pagotto et al. 26 (Suppl 3) J Cardiovasc Pharmacol, S104-6 (1995). However, the central administration of a highly selective ET_{B} receptor agonist, IRL-1620, does not produce any effect on the cardiovascular system, and the systemic and regional circulatory effects of centrally administered ET-1 have been shown to be mediated through the ET_{A} receptors. Gulati et al. 26(Suppl 3) J. Cardiovasc Pharmacol S244-6 (1995); Rebello et al. 676(1) Brain Res 141-50 (1995).

Intracerebroventricular administration of ET-1 produces a transient rise followed by sustained fall in the mean arterial blood pressure (BP). Gulati et al. 58(5) Life Sci, 437-45 (1996). The pressor effect was accompanied by an increase in renal sympathetic nerve activity and plasma levels of catecholamines and arginine-vasopressin (Matsumura et al. 17(6.2) Hypertension 1192-96 (1991).

It also has been shown that the effects of central administration of ET-1 are mediated through activation of the sympathetic nervous system because these effects were attenuated by ganglion blockers. Kawano et al. 7(6) J Hypertens Suppl S22-3 (1989); Matsumura et al. 17(6.2) Hypertension 1192-96 (1991). Intracisternal administration of ET-1 elicited a transient increase in BP, renal sympathetic nerve activity, and phrenic nerve activity. A subsequent fall in BP was accompanied by a decrease in renal sympathetic nerve activity and phrenic nerve activity. Kuwaki et al. 44(1) Jpn J Physiol 1-18 (1994). The observation that central ET-1 induced increase in pressor response was suppressed by pretreatment with phenoxybenzamine. Ouchi et al. 256(6.2) Am J Physiol H1747-51 (1989), farther implicates the active participation of sympathetic nervous system in the initial pressor phase.

Evidence exists that a central ET mechanism is involved in the actions of morphine. It has been found that ET antagonists, including BQ123, for example, can potentiate morphine-induced analgesia and hyperthermia without affecting catalepsy. It is possible influence the potentiating the effects of an opiate analgesic, like morphine, by administration of a therapeutically effective amount of an endothelin receptor antagonist. See U.S. Patent Application Pub. No. 2003/0100507.

The involvement of central ET receptors in morphine analgesia and tolerance has been reported in adult rats, in which it was found that ET_{A} receptor antagonists significantly potentiated morphine analgesia, (Bhalla et al. 23(10) Peptides 1837-45 (2002)) and restored analgesic response of morphine during tolerance (Bhalla et al. 24(4) Peptides 553-61 (2003)).

Although peripheral ET_{B} receptor agonist, IRL1620, has been shown to be involved in analgesia through opioid receptors, centrally administered ET_{B} receptor agonists did not produce any effect on morphine analgesia. Bhalla et al. 72(1) Pharmacology 20-25 (2004). Studies conducted in neonatal rats showed involvement of ET receptors in morphine tolerance: ET receptor antagonists did not act on opioid receptors directly, but modulated the action of morphine by acting through G-proteins. Puppala et al. 86(2) Biol Neonate 138-44 (2004). It has been shown that chronic administration of morphine leads to modulation of Gi/Go-coupled receptors. In contrast, corresponding changes in the ET system and involvement of ET receptors in morphine withdrawal in the brain of neonatal rats remained unknown before the present invention. Therefore the present invention evaluates the effect of ET_{A} receptor antagonist and ET_{B} receptor agonist on G-protein stimulation in neonatal rats undergoing morphine withdrawal by [³⁵S]GTPγS binding assay.

Briefly, the present invention provides for a role of central ET receptors in morphine withdrawal in neonatal rats. The underlying aim was to determine if activation of G-proteins coupled to opioid and ET receptors by morphine and various ET receptor modulators is affected during morphine withdrawal in neonatal rats. Pregnant female rats were rendered tolerant to morphine by chronic exposure to morphine pellets over seven days. On day eight, pellets were removed and rats were allowed to undergo withdrawal for 24 hours. Rat pups were delivered by cesarean section. G-protein stimulation induced by morphine; ET-1; ET_{A} receptor antagonist, BMS182874; and ET_{B} receptor agonist, IRL1620, were determined in the brain of neonatal rats undergoing morphine withdrawal by [³⁵S]GTPγS binding assay. Morphine produced higher (P<0.05) maximal stimulation of G-protein in morphine withdrawal group (83.60%) compared to placebo group (66.81%). ET-1-induced G-protein stimulation was also altered, and EC₅₀ during morphine withdrawal (170.60 nM) was significantly higher than placebo (62.5 nM, P<0.05). ET_{A} receptor antagonist, BMS182874-induced maximal stimulation in morphine withdrawal group (86.07%, EC₅₀=31.25 nM) was significantly higher than placebo group (EC₅₀>1000 nM). ET_{B} agonist, IRL1620-induced G-protein stimulation was similar in placebo (73.43%, EC₅₀=13.26 nM) and morphine withdrawal groups (75.08%, EC₅₀=11.70 nM), respectively. The invention herein provides for the involvement of central ET_{A} receptors in neonatal morphine withdrawal.

Mechanisms involved in withdrawal after chronic opioid administration involve changes in opiate signal transduction and interactions between opiate and non-opiate systems. Vaccarino & Kastin 22(12) Peptides 2257-328 (2001). The present invention provides information on the effect of morphine withdrawal on G-protein activation of opioid and ET receptors in the brain of neonatal rats.

Studies have shown that infant rat pups and fetal rats experience opiate tolerance if dams are exposed to opiates during pregnancy. Barr et al. 60(1) Pharmacol Biochem Behav 97-104 (1998). The invention of the present invention shows that morphine induced stimulation of GTP binding was higher while ET-1 induced stimulation of GTP binding was lower in neonatal rats undergoing morphine withdrawal compared to placebo. These findings clearly implicate a role of central ET receptors in morphine withdrawal in neonatal rats. It was further found that ET_{A} receptor antagonist, BMS182874, did not affect GTP binding in normal but significantly increased GTP binding in morphine withdrawal neonatal rats. In other words, morphine withdrawal may not significantly alter ET receptor activation, but G-proteins coupled to ET receptors may be involved in morphine withdrawal. Our results indicate that these changes taking place in G-protein coupling mechanisms are restored by ET_{A} receptor antagonists.

Although opioid receptor and ET receptor coupling to G-proteins is affected in a similar manner in morphine tolerance, the present findings suggest that opioid receptors and ET receptors are affected differently during withdrawal. The development of opioid dependence and withdrawal may involve complex interactions between several neurotransmitter systems having opposing actions on the G-protein system. Basheer et al. 70(1) Brain Res Dev Brain Res 145-48 (1992). Neonates have been found to have significantly different characteristics of receptors and concentration of neurotransmitters compared to adults, and extensive postnatal developmental changes take place in the CNS. Based on the findings of the Example, below, ET_{A} receptors may play a role in morphine withdrawal in neonatal rats. Because ET_{A} receptor antagonists restore coupling of G-proteins to opioid receptors, this may be clinically significant in the management of opiate tolerance and withdrawal syndrome.

### EXAMPLES

### Example 1

### Action of Et Receptors in Neonatal Rats Pups Undergoing Morphine Withdrawal

Neonatal rats harvested from pregnant female Sprague-Dawley rats (Harlan, Indianapolis, Ind.) at term (day twenty-two of gestation) were randomly selected from each litter and used for [³⁵S]GTPγS binding. Studies were conducted according to guidelines established by Animal Care Committee of University of Illinois at Chicago.

Morphine and placebo pellets were obtained from National Institute of Drug Abuse, Rockville, Md. Guanosine-5′-diphosphate (GDP) was dissolved in assay buffer (Sigma Aldrich, St. Louis, Mo.). [³⁵S]GTPγS (1000 Ci/mmol) (Amersham Pharmacia Biotech, Piscataway, N.J.) was diluted in assay buffer. Unlabeled guanosine-5′-o-(3-thio)triphosphate (GTPγS) was dissolved in assay buffer (Sigma Aldrich, St. Louis, Mo.). Morphine sulfate (Mallinckrodt Chemical Co., St. Louis, Mo.) and IRL1620, (Sigma Aldrich, St. Louis, Mo.) were dissolved in sterile saline. BMS182874 (Tocris Cookson Inc., Ellisville, Mo.) was dissolved in 20% dimethylsulfoxide. ET-1 (American Peptides Company Inc., Sunnyvale, Calif.) was dissolved in 0.1% bovine serum albumin Dilutions of all drugs were prepared in assay buffer.

Pregnant female rats were rendered tolerant to morphine by pellet implantation procedure. Gulati & Bhargava 27(12) Neuropharmacology 1231-37(1988). Rats were divided into two groups: Group 1 received placebo pellets (N=4); group 2 received morphine pellets (N=4). Each rat was subcutaneously implanted with 6 pellets during a seven-day period. One morphine pellet was implanted on day fourteen of gestation, two pellets on day sixteen of gestation, and three pellets on day eighteen of gestation. Control rats received placebo pellets containing the same excipients without morphine. On day twenty-one of gestation, both placebo and morphine pellets were removed, the incision was closed with sterile wound clips and rats were allowed to undergo withdrawal from morphine for 24 hours. Pregnant females on day twenety-two of gestation (at term) were anesthetized using 1% isoflurane anesthesia and rat pups were delivered by cesarean section. Pups were sacrificed immediately, cerebellum was removed, and brain was stored at −70° C. until analyzed. Neonatal rats from both groups were randomly selected and used for [³⁵S]GTPγS binding in neuronal membranes.

[³⁵S]GTPγS binding was performed according to the procedure described earlier (Narita et al. 913(2) Brain Res 170-73 (2001)) using approximately 100 μg protein in each sample. Total volume in each tube was 0.5 ml, containing 0.35 ml of homogenate, various concentrations of drugs (morphine, ET-1, BMS182874, and IRL1620), 30 μM GDP, 100 nM [³⁵S]GTPγS, and assay buffer. Concentration range for morphine, ET-1, BMS182874, and IRL1620 was 0.98 to 1000 nM. Non-specific binding was measured using 10 μM unlabeled GTPγS. Specific binding was expressed as fmnol/mg protein (mean±SEM).

Data was analyzed by one-way ANOVA followed by Bonferroni test. A level of P<0.05 was considered significant.

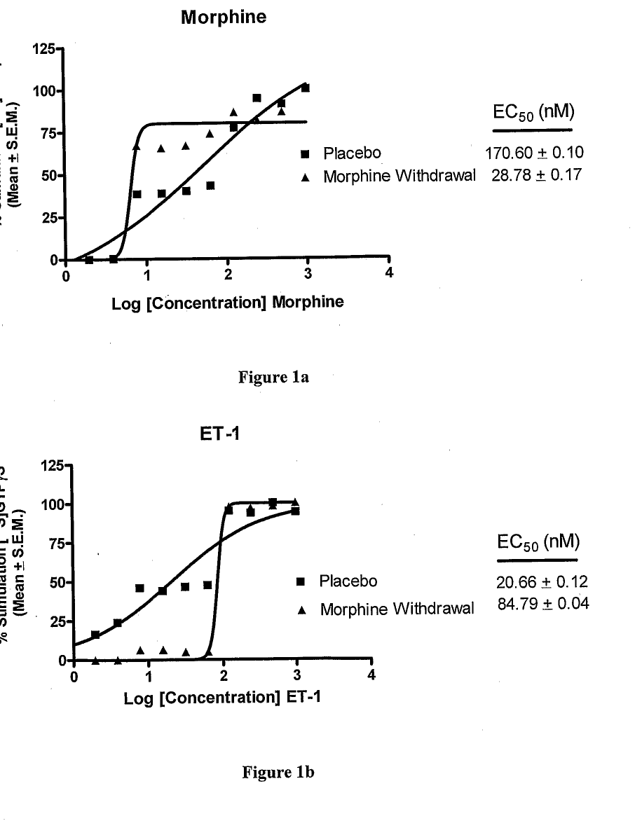

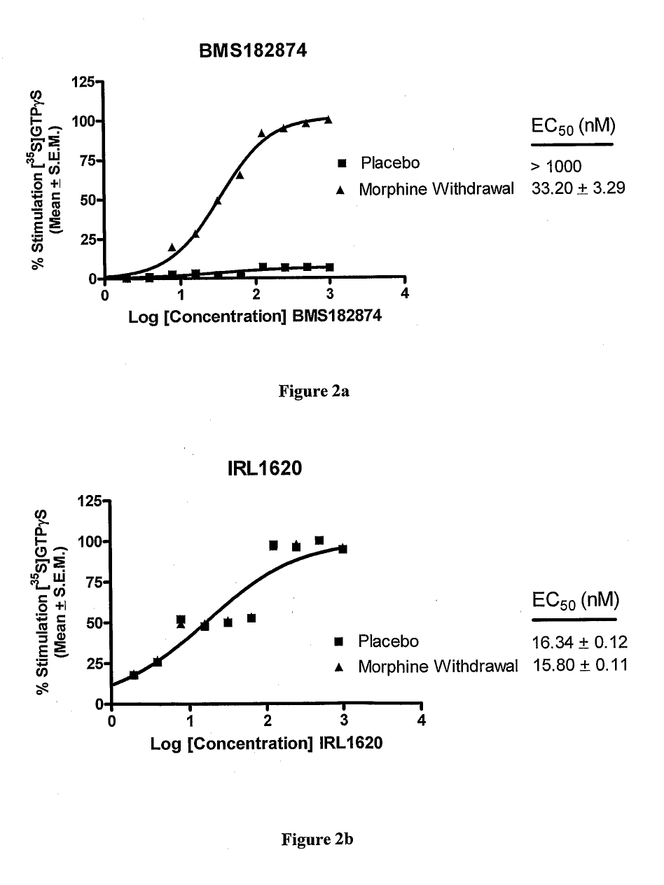

Percent stimulation of [³⁵S]GTPγS binding is shown in FIGS. 1 and 2. Morphine and ET-1 produced concentration-dependent increase in [³⁵S]GTPγS binding. Basal GTP binding was in the range of 0.14±0.01 to 0.89±0.40 fmol/mg protein. Basal binding was statistically similar (P>0.05) in placebo and morphine withdrawal groups. Maximal GTP binding in morphine withdrawal group (83.60±5.77%) was significantly higher (P<0.05) as compared to placebo (66.81±1.51%) (FIG. 1a). EC50 value for morphine-stimulated GTP binding in morphine withdrawal group was 28.78±0.17 nM. This was significantly lower than EC50 value in placebo group (170.60±0.10 nM). ET-1 induced GTP binding was attenuated in the morphine withdrawal group, however at concentrations greater than EC50 value; G-protein activation was increased. Maximal stimulation in the placebo and morphine withdrawal group was 74.88±1.50% and 87.16±4.83% respectively (FIG. 1b). EC50 value in morphine withdrawal group was 84.79±0.04 nM. This was significantly higher than EC50 value in placebo group (20.66±0.12 nM).

Maximal GTP binding with ET_{A} receptor antagonist, BMS 182874, in placebo group was 6.71±1.51%. This indicates that BMS182874 did not stimulate G-proteins in the placebo group. In morphine withdrawal group, BMS182874 produced a maximal stimulation of 79.90±8.22%, which was significantly higher (P<0.05) compared to placebo (FIG. 2a). EC50 value for BMS182874-stimulated [³⁵S]GTPγS binding in placebo group was greater than 1000 nM. EC50 value (33.20±3.29 nM) in morphine withdrawal group was significantly lower (P<0.05) compared to placebo. ET_{B} receptor agonist, IRL1620, produced a maximal stimulation of 73.43±8.89% in placebo group, which was similar (P>0.05) to morphine withdrawal group (75.08±5.41%) (FIG. 2b). EC50 values for IRL1620-stimulated [³⁵S]GTPγS binding in placebo (16.34±0.12 nM) and morphine (15.80±0.11 nM) withdrawal groups were also similar.

What is claimed is:

1. A method for managing opioid tolerance or withdrawal, the method comprising administering an effective amount of an ET receptor antagonist.

2. A method for regulating G-protein coupling to opioid receptors in an individual experiencing opioid tolerance or withdrawal, the method comprising administering an effective amount of an ET receptor antagonist.

3. A method for identifying a novel ET receptor antagonist, the method comprising assessing the G-protein coupling activity to ET receptors in a biological sample obtained from an individual experiencing opioid tolerance or withdrawal.

4. A method for managing opioid tolerance or withdrawal, the method comprising assessing the G-protein coupling activity to ET receptors in a biological sample obtained from an individual experiencing opioid tolerance or withdrawal.
